(19) United States
(12) Patent Application Publication
(10) Pub. No.: US 2004/0043919 A1
(43) Pub. Date: Mar. 4, 2004

(54) Method for the treatment of neurological and neuropsychological disorders

(76) Inventors: Stephan Von Horsten, Wedemark (DE); Torsten Hoffmann, Koernerstrasse (DE); Hans-Ulrich Demuth, Halle/Saale (DE); Kerstin Kuhn-Wache, Halle (DE); Daniel Friedrich, Halle/Saale (DE)

Correspondence Address: BROWN, RUDNICK, BERLACK & ISRAELS, LLP., BOX IP, 18TH FLOOR, ONE FINANCIAL CENTER, BOSTON, MA, 02111, US

(21) Appl. No.: 10/415,263

(22) PCT Filed: Oct. 29, 2001
(86) PCT No.: PCT/EP01/12478

(51) Int. Cl. A01N 61/00; A61K 38/10

(52) U.S. Cl. 514/1; 514/2

(57) ABSTRACT

The present invention discloses a method for therapeutically treating an animal, including a human, for psychosomatic, depressive and neuropsychiatric diseases, such as anxiety, depression, insomnia, schizophrenia, epilepsy, spasm and chronic pain. Administration of a suitable attractin inhibitor causes the reduction of activity in the enzyme attraction or in isoforms thereof in the brain of mammals and leads as a causal consequence to a reduced degradation of the neuropeptide Y (NPY) and similar substrates. Such treatment will result in a reduction or delay in the decrease of the concentration of functionally active neuronal NPY (1-36). As a consequence of the resulting enhanced stability of the endogenous NPY (1-36), NPY activity is prolonged thereby resulting among other things in functionally active NPY Y1 receptor activity thereby facilitating antidepressive, anxiolytic, analgesic, t antihypertension and other neurological effects.

### BACKGROUND OF THE INVENTION

1. Field of the invention

The present invention relates to the function of attractin and of attractin isoforms within the central nervous system (CNS) and their biological effects on neuropeptide levels, neurotransmission and behavior. The present invention also relates to the potentiation of endogenous neurological and neuropsychological effects of brain neuropeptide Y (NPY) systems and other substrates of attractin by selective inhibition of attractin and of attractin isoforms. The invention relates further to the treatment of hypertension, fever, sleep dysregulation, anorexia, anxiety related disorders including depression, seizures including epilepsy, drug withdrawal and alcoholism, neurodegenerative disorders including cognitive dysfunction and dementia, and neuropsychiatric disorders including schizophrenia, via a potentiation of NPY Y 1 receptor mediated effects resulting from an inhibition of attractin and of attractin isoforms within the CNS.

2. Background Art

Discovery of NPY

Neuropeptide Y SPY), a 36 amino acid peptide belonging to the pancreatic polypeptide family, was first isolated from porcine brain in 1982 (Tatemoto and Mutt, 1982). NPY is present in all sympathetic nerves innervating the cardiovascular system and is the most abundant peptide in the brain and the heart. Additionally, in rats, but not in humans, NPY is also found extraneuronally in platelets and endothelium (Zukovska-Grojec et al., 1993). Originally, NPY was known as a potent vasoconstrictor and a neuromodulator. Released by stress, exercise, and myocardial ischemia, NPY has been implicated in coronary heart disease, congestive heart failure, and hypertension (Zukovska-Grojec et al, 1998). More recently, because of the potent ability of NPY to stimulate food intake, it is suspected to play a role in obesity and diabetes (Kalra et al., 1999). Latest findings indicate that NPY is also a mitogen for rat aortic vascular smooth muscle cells (Zukovska-Grojec et al., 1999).

NPY-related research has focussed on at least three main directions: (1) Co-transmission and sympathetic vasoconstriction, because of its co-expression with noradrenaline; (2) neurotransmission and function within the CNS, because of potent consummatory effects; and (3) evolution of NPY, since NPY is one of the most highly conserved bio-active peptides known (Colmers and Wahlestedt, 1993; Lundberg, 1996; Wahlestedt and Reis, 1993; Wettstein et al., 1996). NPY acts on at least six receptors (Y1-Y6), with varying peptide pharmacology and distinct distribution in the CNS (Gehlert, 1998) (Tab. 1).

Distribution of NPY, NPY Receptor Subtypes and mRNA

The distribution of NPY itself, NPY receptor protein and their mRNA within the CNS of human and rat brains has recently been reviewed (Dumont Y, Jacques D, St-Pierre, J. -A., Tong, Y., Parker, R., Herzog H. and Qurion, R., 2000; in Handbook of Chemical Neuroanatomy, Vol. 16: Peptide Receptors, Part I; Quirion, R., Björklund, A. and Hökfeld, T., editors). A brief survey is given in Tab. 1.

NPY-containing neurons are evident in the nasal mucosa of various species including man, often associated with glandular acini and blood vessels (Baraniuk et. Al., 1990; Grunditz et. al., 1994). Stimulation of the parasympathetic nerve supply to the nasal mucosa (vidian nerve) in dogs increases blood flow in the region and causes mainly atropine resistance. Intravenous administration of NPY reduces vasodilitation due to parasympathetic nerve stimulation, an effect that was not mimicked by the NPY Y1-selective agonist [Leu31, Pro34]NPY, but was mimicked by administration of the NPY Y2- receptor agonist N-acetyl[Leu28,Leu31JNPY(24-36) (Lacroix et al., 1994). This is consistent with a prejunctional NPY Y2- like receptor-mediated inhibition of transmitter release from parasympathetic nerve terminals.

NPY Receptor Function

NPY is unarguably the most abundant neuropeptide discovered to date, with a wide distribution in the CNS and the peripheral nervous system (PNS). NPY forms a family of peptides together with peptide YY (PYY) (approximately 70% homology) and pancreatic polypeptide (PP) (approximately 50% homology); both NPY and PYY are extremely bio-active, whereas PP is generally much less active (Gehlert, 1998; Wahlestedt and Reis, 1993) (Tab. 2).

Two receptor subtypes of NPY have been called neuropeptide Y Y1 (postjunctional) and neuropeptide Y Y2 (prejunctional) on the basis of the different responses to a truncated analog of the related peptide YY-(13- 36), when compared with neuropeptide Y in in vitro assay systems (Wahlestedt et al., 1986). Activation of neuronal prejunctional NPY receptors generally inhibits nerve activity, reducing the release of neurotransmitters in response to nerve impulses and in response to local factors acting to release neurotransmitters (Wahlestedt et al., 1986). The prejunctional or neuropeptide Y Y2 receptor classification was based on actions of peptide YY (13-36) but in many systems this molecule, as well as neuropeptide Y-(13-36), does exhibit pressor activity (Rioux et al., 1986; Lundberg, et al., 1988; Potter et al., 1989). This has been interpreted by some to indicate that in some vascular beds there are two types of neuropeptide Y receptors (both neuropeptide Y Yj and neuropeptide Y2) on postjunctional membranes (Schwartz et al., 1989). However the lack of selectivity of these molecules may be due to retention of partial agonistic activity on Yj receptors, which permits them to evoke a reduced functional response. Previously, a 13-36 analog of neuropeptide Y, (Leu 17, Glu″, Ala 21, Ala 22, Glu 23, LeU28, LeU31) neuropeptide Y-(13-36) (ANA neuropeptide Y-(13-36)) which displayed prejunctional activity equivalent to the whole neuropeptide Y molecule in studies in vivo was described (Potter et al., 1989).

Apart from these historically well-defined neuropeptide Y receptors the existence of a number of other subtypes (Y3, Y4, Y5 and Y6) has been suggested on a pharmacological basis (Michel et al., 1998) and details of the cloning of receptors corresponding to Y1, Y2, Y4 and Y5 have been published (Herzog et al., 1992; Gerald et al., 1995; Bard et al., 1995; Gerald et at., 1996) (Tab. 1). The distribution and physiological significance of these various receptor subtypes has yet to be defined. Although some controversy has existed about the selectivity of truncated forms of neuropeptide Y for one or other receptor subtype (Potter et al., 1989), the emerging picture supports the initial classification into pre- and postjunctional receptor subtypes. Cell lines have been developed which express specifically one neuropeptide Y receptor subtype and the development of receptor-selective analogs of neuropeptide Y has focussed mainly on binding characteristics in these cell lines (Sheikh et al., 1989; Aakerlund et al., 1990; Fuhlendorff et al., 1990). More recently, a cDNA encoding the neuropeptide Y Y1 receptor has been cloned and cell lines expressing the cloned receptor have been analyzed for both specific binding of neuropeptide Y analogs (Herzog et al., 1992) and functional responses elicited by specific analogs. From such binding studies, combined with subsequent studies in vivo, two analogs have been classified as acting specifically on the postjunctional neuropeptide Y Y1 receptor. These neuropeptide Y Y receptor selective analogs, (Pro 34) neuropeptide Y and (Leu″, Pro 34 ) neuropeptide Y, mimic the action of neuropeptide Y in raising blood pressure, and also share similar binding to cell lines expressing only neuropeptide Y Y receptors e.g. the human neuroblastoma cell line SK-N-MC and fibroblast lines expressing the cloned neuropeptide Y Y, receptor (Herzog et al., 1992). Neither exhibits the neuropeptide Y Y2 receptor action an inhibition of cardiac vagal action in vivo, a manifestation of inhibition of acetylcholine release (Potter et al., 1991; Potter and McCloskey, 1992).

TABLE 1
DISTRIBUTION AND FUNCTION OF NPY RECEPTOR SUBTYPES
WITHIN THE CNS

| Recep- | CNS | | | Selective |
|---|---|---|---|---|
| tor- | Expres- | | Selective | Antagonist or |
| subtype | sion | Function | Agonist | selectivity |
| Y1 | Cortex, | Anxiolysis, | Intact N - | BIBP3226; |
| | etc. | LHRH | Terminus: | BIBO 3304 |
| | | Release | [Leu31, | |
| | | | Pro34]NPY | |
| Y2 | Hippo- | Antiamnestic | C-terminale | T4[NPY(33-36)]4; |
| | campus, | | End: | BIIE0246 |
| | Hypo- | | PYY3-36; | |
| | thalamus | | PYY13-36 | |
| Y3 | Nol. | Bradycardia, | NPY >> PYY, | PYY - Insensitivity |
| | Tractus | Hypo- | [Leu31, | |
| | Solitarius | tension | Pro34]NPY | |
| | (NTS) | | | |
| Y4 | Dorsal | Emetic | PP >> NPY, | PP - Preferring |
| | vagal | | PYY | |
| | Complex | | | |
| | (DVC) | | | |
| Y5 (a) | Hypo- | Feeding | NPY, PYY, | [Leu31, Pro34]NPY - |
| | thalamus | | [Leu31, | sensitive, BIBP3226 - |
| | | | Pro34]NPY | non-reversible |
| Y5 (b) | Hypo- | ?; species | ? | ? |
| or Y6 | thalamus | specific | | |

Tab. 1: NPY Receptor subtypes within the CNS; ? = unknown or not investigated

The development of the high affinity, non-peptide NPY antagonists, BIBP3226 and BIBO3304, has facilitated the functional characterization of NPY receptors, as this compound shows selectivity for Y1R, being devoid of activity on at least Y2R, Y3R and Y4R (Doods et al., 1996). Recently, a two Y2 receptor antagonist has been described. One is a TASP-molecule (Grouzmann et al., 1997), the other a non-peptide antagonist (Wieland et al., 1999) and other non-peptide receptor specific compounds became available (Daniels et al., 1995). Thus, specific receptor blockade within the brain would allow the functional characterization of behavioral and physiological effects mediated by central NPY receptors. In addition, mice lacking the Y1R were generated and are available (Pedrazzini et al., 1998). Neurons showing NPY-like immunoreactivity and NPY receptor expression are abundant in the CNS (Tab. 1), and perhaps are most notably found in hypothalamic and so-called limbic structures, but are also co-localized with brain stem monoaminergic neurons and cortical GABA-ergic neurons (Chronwall, 1985; Dumont et al., 1996).

TABLE 2
RECEPTOR SUBTYPES AND PEPTIDE SELECTIVITY

| Receptor subtype | Peptide Potency |
|---|---|
| Y1-like | |
| Y1 | NPY = PYY = Pro³⁴-NPY > PP > NPY₁₃₋₃₆ |
| Y4 | PP >> NPY = PYY = LP-NPY > NPY₁₃₋₃₆ |
| Y6 | NPY = PYY = Pro³⁴-NPY > NPY > NPY₁₃₋₃₆ > PP |
| Y2-like | |
| Y2 | NPY = PYY = NPY₁₃₋₃₆ > Pro³⁴-NPY > PP |
| Y5-like | |
| Y5 | NPY = PYY = Pro³⁴-NPY > NPY¹³⁻³⁶ > PP |
| Not cloned | |
| PP receptor | PP >> PYY = NPY |
| Y3 | NPY = Pro³⁴-NPY = NPY₁₃₋₃₆ >> PYY |
| PYY-preferring | PYY > NPY >> NPY₁₃₋₃₆ >> Pro³⁴-NPY |

Tab. 2: Receptor subtypes and peptide selectivity according to Gehlert, 1998.

NPY, Anxiety and Depression

Anxiolytic-like effects of NPY have been demonstrated using the elevated plus maze test (Montgomery), the punished drinking test (Vogel), and the punished responding test (Geller-Seifter), with potency and efficacy matching those of benzodiazepines (Griebel, 1999; Heilig et al., 1989; Wettstein et al., 1995). NPY acts anxiolytic-like on the response to novelty (Heilig and Murison, 1987; von Hörsten et al., 1998b), and produces anxiolytic-like effects on the elevated plus maze and other anxiety related tests (Wahlstedt and Reis, 1993; Wahlestedt et al., 1993). Interestingly, Y1 receptor antisense-treated rats showed marked anxiety-related behaviors, without alterations of locomotor activity and food intake (Wahlestedt et al., 1993). Additionally, in the Flinder rat strain, a genetic model of depression, Y1 receptor mRNA expression was decreased in different cortical regions and the dentate gyrus of the hippocampus, while Y2 receptor mRNA expression did not differ from controls (Caberlotto et al., 1998). Olfactory butbectomy in the rat has been developed as a model of depression (Leonard and Tuite, 1981). In this model, most of the changes resemble those found in depressed patients (Song et al., 1996). A 7-day i.c.v. administration of NPY in olfactory bulbectomized rats attenuated behavioral and neurotransmitters deficits in this model (Song et al., 1996). NPY Y1, Y2, and possibly Y5 receptors, seem to be involved in the regulation of anxiety levels in rodents, with Y 1-mediated effects being best characterized (Heilig et al., 1993; Kask et al., 1998b). It can be concluded, therefore, that endogenous NPY counteracts stress and anxiety (Heilig et al., 1994). Furthermore, these data suggest that the Y1receptor subtype could be implicated in anxiety- and depression-related behaviors. Additionally, Kask et al. (1996) reported that i.c.v. injection of the Y1 antagonist. BIBP3226, produced anxiogenic-like effects in the elevated plus-maze test, without any locomotor deficit. This effect can be reproduced by the administration of BIBP3226 in the dorsal periaqueductal gray matter but not in the locus coeruleus or the paraventricular nucleus of the hypothalamus (Kask et al., 1998c). Moreover, BIBP3226 and GR231118 administered into the dorsal periaqueductal gray matter decreased the time spent in active social interaction in rats (Kask et al., 1998d). The brain regions which are important for the anti-stress action of NPY include but may not be limited to the amygdala (Sajdyk et al., 1999, Thorsell et al., 1999), locus coeruleus (Kask et al., 1998c) and dorsal periaqueductal gray (Kask et al., 1998a,b). Amygdala NPY is not released under low stress conditions since blockade of NPY Y₁R with BIBP3226 or BIBO3304 did not increase anxiety as measured in the elevated plus-maze and social interaction tests (Kask et al., 1998b; Sajdyk, 1999). Constant NPY-ergic tone, however, seems to exist in the dorsal periaqueductal gray matter, where the NPY Y₁R antagonist had anxiogenic like effects in both experimental anxiety models (Kask et al., 1998a,b). Thus, in certain brain regions, there may be a tonic regulation of anxiety via NPY systems.

Neurological and Psychophysiological Effects of CNS NPY Systems: Pleiotrophy

Thus, numerous studies have addressed the physiological functions of NPY and its congeners in the CNS (for reviews see: Kalra and Crowley, 1992; Dumont et al., 1992; Stanley, 1993; Wahlestedt and Reis, 1993; Grundemar et al, 1993; Gehlert, 1994, 1998; Colmers and Bleakman, 1994; Wettstein et al, 1995; Heilig and Widerlow, 1995; Munglani et al., 1996; Inui, 1999; Bischoff and Michel, 1999; Vezzani et al., 1999) and demonstrated a broad range of effects. No pharmacological approaches exist, at present, to gain advantage of these various physiological functions.

Current Problems in the Treatment of Anxiety Related Disorders using Benzodiazepines or NPY

The current methods for treatment of anxiety are accompanied by several problems:

The benzodiazepines that are commonly used as anxiolytic agents are unnatural compounds with a low or no selectivity. Beside their anxiolytic activity, the benzodiazepines show sedative and anti-epileptic effects and are suspected to influence muscle relaxation. Unfortunately, they are associated with a number of unwanted side effects, namely tiredness, sleepiness, lack of concentration, reduction of attentiveness and reactivity. Chronic application of benzodiazepines causes neurological disorders, like ataxia, dizziness, reflex loss, muscle and language disorders. A long-term treatment with benzodiazepines is predicted to entail dependency and addiction.

The direct i.c.v. administration of neuropeptide Y for the long-term treatment of anxiety in patients is not feasible.

### SUMMARY OF THE INVENTION

It is an object of the present invention to provide a medicament beneficial for neurological and psychophysiological effects. It is especially an object of the present invention, to provide an inhibitor of attractin or of an attractin isoform for the production of a medicament for modulating behavioral and/or neurological adaptive responsiveness to stress including anxiety.

In addition, it is an object of the present invention to overcome or reduce the above stated problems of the prior art by providing a pharmacological approach and results in a maintained or prolonged activity and/or effect of NPY in the brain of mammals.

These objects are solved by the use of an inhibitor of attractin or of attractin isoforms for the production of a medicament for modulating behavioral and/or neurological responsiveness to stress including anxiety.

This results in the magnification of endogenous neurological or neuropsychological effects mediated by NPY Y1 receptors, including but not limited to a reduction of anxiety, treatment of hypertension, fever, sleep dysregulation, anorexia, anxiety related disorders including depression, seizures including epilepsy, drug withdrawal and alcoholism, neurodegenerative disorders including cognitive dysfunction and dementia, and neuropsychiatric disorders including schizophrenia diagnosed in a subject.

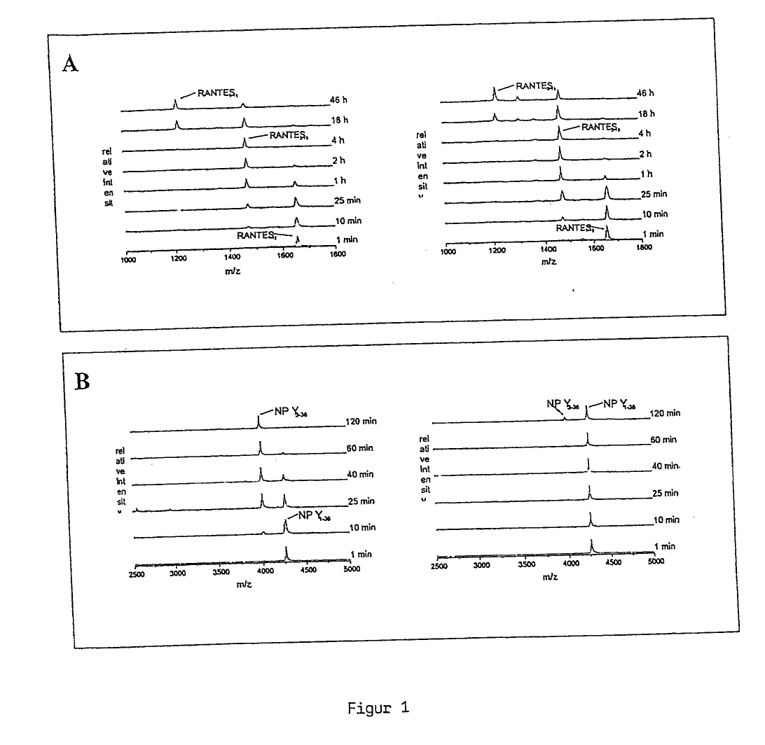

FIG. 1 shows MALDI-TOF mass spectra of the proteolytic processing of RANTES 1-15 by DP IV and attractin (A) and NPY by attractin in absence (left side) and presence (right side) of isoleucyl-thiazolidine hemifumarate (P32/98).

### DETAILED DESCRIPTION OF THE INVENTION

In contrast to other proposed methods in the art, the present invention provides an orally available therapy with low molecular weight inhibitors of attractin or attractin isoforms (isoenzymes). The instant invention represents a novel approach for the treatment of anxiety and other neurological or psychological disorders in mammals. It is user friendly, commercially useful and suitable for use in a therapeutic regime, especially concerning human disease.

Examples for orally available low molecular weight inhibitors of the attractin enzyme activity are agents such as, N-(N′-substituted glycyl)2-cyanopyrrolidines, L-threo-isoleucyl thiazolidine, L-allo-isoleucyl thiazolidine, L-threo-isoleucyl pyrrolidine, L-allo-isoleucyl thiazolidine, and L-allo-isoleuycl pyrrolidine. They are described in U.S. Pat. No. 6,001,155, WO 99/61431, WO 99/67278, WO 99/67279, DE 198 34 591, WO 97/40832, DE 196 16 486 C 2, WO 98/19998, WO 00107617, WO 99/38501, and WO 99/46272, the teachings of which are herein incorporated by reference in their entirety.

Attractin is an enzyme that is an exopeptidase, which selectively cleaves peptides after penultimate N-terminal proline and alanine residues. Presently 5 isoforms of attactin are known. Attractin-like enzymes, which can also be used according to the present invention, can, e.g., be selected by subjecting peptidases to a test for selectivity cleaving peptides after penultimate N-terminal proline and alanine residues, selecting a peptidase which effects such a cleavage and isolating the peptidase.

Various effects of attractin inhibitors imply their impact on normal healthy tissues and organs, when they are used for the treatment of a pathologically altered tissue. The goal of the present invention is the development of highly selective brain targeted inhibitors for attractin and of attractin isoforms, which display a high bioavailability and an exactly predictable activity time in the target tissue.

Examples for orally available low molecular weight agents are prodrugs of stable and unstable inhibitors of the attractin enzyme activity which comprise the general formula A-B-C, whereby A represents an amino acid, B represents the chemical bond between A and C or an amino acid, and C represents an unstable or a stable inhibitor of the attractin enyzme activity, respectively. They are described in WO 99/67278, WO 99/67279 the teachings of which are herein incorporated by reference in their entirety.

The present invention relates to a novel method in which the reduction of activity of the enzyme attractin or of attractin isoforms in the brain of mammals induced by effectors of the enzyme leads as a causal consequence to a reduced degradation of the neuropeptide Y (NPY). Such treatment will result in a reduction or delay in the decrease of the concentration of functional active NPY (1-36).

According to the present invention it has been found that the effect and/or activity of NPY in the brain of mammals, especially humans, can be maintained or prolonged by the administration of inhibitors of attractin or of attractin isoforms (isoenzymes). Thereby, the degradation of NPY in the brain can be reduced. This results in an alleviation or improvement of psycosomatic, depressive and/or neuropsychiatric diseases. The instant invention especially represents a novel approach for the treatment of anxiety and other neurological or psychological disorders. It is user friendly, commercially useful and suitable for use in a therapeutic regime, especially concerning human disease.

Surprisingly, the inventors have found that the administration of the attractin inhibitor isoleucyl thiazolidine exhibits an anxiolytic effect.

Attractin and its isoforms are present and widely distributed in rat brain (Lu et al., 1999). The inventor shows in example 1, that NPY is a principal substrate for attractin in vitro.

As a consequence of the resulting enhanced stability of the endogenous NPY (1-36) caused by the inhibition of attractin activity, NPY activity is prolonged resulting in functionally active NPY Y 1 receptor activity facilitating—among others—anti-depressive, anxiolytic and anti-hypertensive effects (see above).

The method of the present invention for treating anxiety in an animal, including humans, in need thereof, comprises potentiating NPY's presence by inhibiting attractin or attractin isoforms. Oral administration of an attractin inhibitor may be preferable in most circumstances. By inhibiting the attractin enzyme activity, the half-life of the active form of NPY will be appreciably extended and maintained under physiological conditions. The extended presence of active NPY will enhance the NPY Y1 receptor activity.

This invention also provides pharmaceutical compositions. Such compositions comprise a therapeutically (or prophylactically) effective amount or the inhibitor (and/or a sugar pill to accompany administration of an attractin inhibitor), and a pharmaceutically acceptable carrier or excipient, especially adapted for targeting the brain. Suitable carriers include but are not limited to saline, buffered saline, dextrose, water, glycerol, ethanol, and combinations thereof. The carrier and composition are preferably produced under good laboratory practices conditions and most preferably are sterile. The formulation is ideally selected to suit the mode of administration, in accordance with conventional practice.

Suitable pharmaceutically acceptable carriers include but are not limited to water, salt solutions (for example, NaCl), alcohols, gum arabic, vegetable oils, benzyl alcohols, polyethylene glycols, gelatin, carbohydrates such as lactose, amylose or starch, magnesium stearate, talc, viscous paraffin, perfume oil, fatty acid esters, hydroxymethylcellulose, polyvinyl pyrrolidone, etc. The pharmaceutical preparations can be sterilized and if desired mixed with auxiliary agents, for example, lubricants, preservatives, stabilizers, wetting agents, emulsifiers, salts for influencing osmotic pressure, buffers, coloring, flavoring and/or aromatic substances and the like which do not deleteriously react with the active compounds, but which improve stability, manufacturability and/or aesthetic appeal.

The compositions, if desired, can also contain minor amounts of wetting or emulsifying agents, or pH buffering agents. In addition, the composition can be a liquid solution, suspension, emulsion, tablet, pill, capsule, sustained release formulation, or powder. In addition, the composition can be formulated as a suppository, with traditional binders and carriers such as triglycerides. Oral formulations can include standard carriers such as pharmaceutical grades of mannitol, lactose, starch, magnesium stearate, polyvinyl pyrrolidone, sodium saccharine, cellulose, magnesium carbonate etc.

Further, the compositions can be formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active compound. Where the composition is to be administered by infusion, it can be dispensed with an infusion bottle containing sterile pharmaceutical grade water, saline or dextrose/water. Where the composition is administered by injection, an ampoule of sterile water for injection or saline can be provided so that the ingredients may be mixed prior to administration.

Finally, compositions of the invention can be formulated as neutral or salt forms. Pharmaceutically acceptable salts include those formed with free amino groups such as those derived from hydrochloric, phosphoric, acetic, oxalic, tartaric acid, etc., and those derived from sodium, potassium, ammonium, calcium, ferric hydroxides, isopropylamine, triethylamine, 2-ethylamino ethanol, histidine, procaine, etc.

The amount of the invention's composition which will be effective in the treatment of a particular disorder or condition will depend on the nature of the disorder or condition, and can be determined by standard clinical techniques. In addition, in vitro and/or in vivo assays may optionally be employed to help identify optimal dosage ranges. The precise dose to be employed in the formulation will also depend on the route of administration, and the seriousness of the disease or disorder, and should be decided according to the judgement of the practitioner and each patient's circumstances.

It will be readily understood by the skilled artisan that numerous alterations may be made to the examples and instructions given herein including the generation of different attractin inhibitors and alternate therapeutic compositions without departing from either the spirit or scope of the present invention.

The present invention will now be illustrated with reference to the following example, focussing on the anxiolytic-like and stress-protective-like action of reduced attractin activity.

### EXAMPLE

### Example 1

NPY is a Substrate for Human Attractin in vitro

Attractin from human plasma (Baxter GmbH Germany, Plasmazentrum Halle) was prepared from 100 ml plasma from healthy humans. Matrix-assisted laser desorption/ionisation mass spectrometry was carried out using the Hewlett-Packard G2025 LD-TOF System.

To obtain spectra of peptides (25 μM) by the treatment of purified DP IV and attractin in the presence or absence of isoleucyl-thiazolidine (10 μM), substrates were incubated at 37° C. with 40 mM tricine/HCl buffer pH 7.6 and enzyme solution in a 2:2:1 ratio. Samples of the reaction mixtures were removed at various time intervals and mixed with equal volumes of the matrix solution. By mixing assay sample and matrix, the low pH of the matrix solution stopped the enzymatic reaction. A small volume of this mixture was transferred to a probe tip and immediately evaporated in a Hewlett-Packard G2024A Sample Prep Accessory.

In further studies the proteolytic activity of attractin has been investigated. The Kₘ value for Gly-Pro-pNA was determined to be 0.14 mM, comparable to values found for DP IV, attractin and a serum DP IV-like activity. In addition, the cleavage of bioactive peptides such as NP Y, RANTES, GIP, and Glucagon by purified attractin has been analyzed. Similar to DP IV, attractin is capable of releasing the N-terminal dipeptide Tyr-Pro from NP Y (FIG. 1B, left side). In presence of isoleucyl-thiazolidine, the cleavage was suppressed (FIG. 1B, right side). Previously, differences within hydrolysis of the chemokine RANTES by DP IV and attractin have been described⁴. However, our results do not confirm these data. Similar to DP IV (FIG. 1A, left side), purified attractin is capable of releasing sequentially the first (Ser-Pro) as well as the second dipeptide (Tyr-Ser) from N-terminus of the synthetic RANTES₁₋₁₅ (FIG. 1A, right side).

References

Aakerlund, L., U. Gether, T. W. Schwartz, and O. Thastrup (1990), Yj receptors for neuropeptide Y are coupled to mobilization of intracellular calcium

Agmo, A. and Belzung, C., Interactions between dopamine and GABA in the control of ambulatory activity and neophobia in the mouse, Pharmacol. Biochem. Behav., 59 (1998) 239-247.

Agmo, A. and Belzung, C., The role of subtypes of the opioid receptor in the anxiolytic action of chlordiazpoxide Neuropharmacology, 37 (1998) 223-232.

Agmo, A., Galvan, A., Heredia, A. and Morales, M., Naloxone blocks the antianxiety but not the motor effects of benzodiazepines and pentobarbital: experimental studies and literature review, Psychopharmacology Berl., 120 (1995) 186-194.

Arvat, E., Maccagno, B., Ramunni, J., Di-Vito, L., Gianotti, L., Broglio, F., Benso, A., Deghenghi, R., Camanni, F. and Ghigo, E., Effects of dexamethasone and alprazolam, a benzodiazepine, on the stimulatory effect of hexarelin, a synthetic GHRP, on ACTH, cortisol and GH secretion in humans, Neuroendocrinology., 67 (1998) 310-316.

Band, L. C., Pert, A., Williams, W., De Costa, B. R., Rice, K. C. and Weber, R. J., Central μ-opioid receptors mediate suppression of natural killer activity in vivo, Prog. Neuro. Endocrinol. Immunol., 5 (1992) 95-101.

Baram, T. Z., Yi, S., Avishai, E. S. and Schultz, L., Development neurobiology of the stress response: multilevel regulation of corticotropin-releasing hormone function, Ann. N.Y. Acad. Sci., 814 (1997) 252-265.

Baraniuk, J. N., Castellino, S., Lundgren, J. D., Goff, J., Mullol, J., Merida, M., Shelhamer, J, H. and Kaliner, M. A. (1990), Neuropeptide Y (NPY) in human nasal mucosa. J. Respir. Cell Mol. Biol. 3, 165-173;

Bard, J. A., Walker, M. W., Branchek, T. A. and Weinshank, R. L. (1995), J. Biol. Chem., 270, 26762-26765. Cloning and functional expression of Y4 subtype receptor for pancreatic polypeptide, neuropeptide Y, and peptide YY.

Berkenbosch, F., van-Oers, J., del-Rey, A., Tilders, F. and Besedovsky, H., Corticotropin-releasing factor-producing neurons in the rat activated by interleukin-1, Science, 238 (1987) 524-526.

Bertolucci, M., Perego, C. and De Simoni, M. G., Central opiate modulation of peripheral IL-6 in rats, NeuroReport, 7 (1996) 1181-1184.

Bertolucci, M., Perego, C. and de Simoni, M. G., Interleukin-6 is differently modulated by central opioid receptor subtypes, Am. J. Physiol., 273 (1997) R956-R959.

Bileviciute, I., Stenfors, C., Theodorsson, E., Beckman, M. and Lundeberg, T., Significant changes in neuropeptide concentrations in the brain of normotensive (WKY) and spontaneously hypertensive (SHR) rats following knee joint monoarthritis, Brain Res., 704 (1995) 71-78.

Britton, D. R., Koob, G. F., Rivier, J. and Vale, W., Intraventricular corticotropin-releasing factor enhances behavioral effects of novelty, Life Sci., 31 (1982) 363-367.

Britton, K. T., Lee, G. and Koob, G. F., Corticotropin releasing factor and amphetamine exaggerate partial agonist properties of benzodiazepine antagonist Ro 15-1788 in the conflict test, Psychopharmacology Berl., 94 (1988) 306-311.

Brown, M. R, Fisher, L. A., Spiess, J., Rivier, J., Rivier, C. and Vale, W., Comparison of the biologic actions of corticotropin-releasing factor and sauvagine, Regul. Pept., 4 (1982) 107-114.

Calenco, C. G., Dauge, V., Gacel, G., Feger, J. and Roques, B. P., Opioid delta agonists and endogenous enkephalins induce different emotional reactivity than mu agonists after injection in the rat ventral tegmental area, Psychopharmacology Berl., 103 (1991) 493-502.

Carr, D. J., Rogers, T. J. and Weber, R. J., The relevance of opioids and opioid receptors on immunocompetence and immune homeostasis, Proc. Soc. F,p. Biol. Med., 213 (1996) 248-257.

Chen, C., Dagnino, R., De-Souza, E. B., Grigoriadis, D. E., Huang, C. Q., Kim, K. I., Liu, Z., Moran, T., Webb, T. R., Whitten, J. P., Xie, Y. F. and McCarthy, J. R., Design and synthesis of a series of non-peptide high-affinity human corticotropin-releasing factor 1receptor antagonists, J. Med. Chem., 39 (1996) 4358-4360.

Chronwall, B. M., Anatomy and physiology of the neuroendocrine arcuate nucleus, Peptides, 6 Suppl 2 (1985) 1-11.

Colmers, W. and Wahlestedt, C. (1993) The biology of neuropeptide Y and related peptides. Humana Press, Totowa, N.J.

Conte, D. B., Rey, M., Boudouresque, F., Giraud, P., Castanas, E., Millet, Y., Codaccioni, J. L. and Oliver, C., Effect of 41 -CRF antiserum on the secretion of ACTH, B-endorphin and alpha-MSH in the rat, Peptides, 4 (1983) 301-304.

Daniels, A. J., Matthews, J. E., Slepetis, R. J., Jansen, M., Viveros, O. H., Tadepalli, A., Harrington, W., Heyer, D., Landavazo, A., Leban, J. J. and et, a., High-affinity neuropeptide Y receptor antagonists, Proc. Natl. Acad. Sci. U.S.A., 92 (1995) 9067-9071.

Devine, D. P., Taylor, L., Reinscheid, R. K., Monsma-F J, J., Civelli, O. and Akil, H., Rats rapidly develop tolerance to the locomotor-inhibiting effects of the novel neuropeptide orphanin FQ, Neurochem. Res., 21 (1996) 1387-1396.

Dieterich, K. D., Lehnert, H. and De-Souza, E. B., Corticotropin-releasing factor receptors: an overview, Exp. Clin. Endocrinol. Diabetes, 105 (1997) 65-82.

Doods, H. N., Wieland, H. A., Engel, W., Eberlein, W., Willim, K. D., Entzeroth, M., Wienen, W. and Rudolf, K., BIBP 3226, the first selective neuropeptide Y1 receptor antagonist: a review of its pharmacological properties, Regul. Pept., 65 (1996) 71-77.

Duke-Cohan J S, Morimoto C, Rocker J A, Schlossman S F: Serum high molecular weight dipeptidyl peptidase IV (CD26) is similar to a novel antigen DPPT-L released from activated T cells, The journal of immunology 156, 1714-21 (1996)

Duke-Cohan J S, Tang W, Schlossman S F: Attractin: A cub-family protease involved in T cell-mono-cyte/macrophage interactions, Adv. Exp. Med. Biol. 477, 173-185 (2000)

Dumont, Y., Fournier, A., St, P. S. and Quirion, R., Autoradiographic distribution of [125I]Leu31,Pro34]PYY and [125I]PYY3-36 binding sites in the rat brain evaluated with two newly developed Y1 and Y2 receptor radioligands, Synapse, 22 (1996) 139-158.

Dunn, A. J., Berridge, C. W., Lai, Y. I. and Yachabach, T. L., CRF-induced excessive grooming behavior in rats and mice, Petides, 8 (l987) 841-844.

Egawa, M., Yoshimatsu, H. and Bray, G. A., Neuropeptide Y suppresses sympathetic activity to interscapular brown adipose tissue in rats, Am. J. Physiol., 260 (1991) R328-R334

Eghbal, A. M., Hatalski, C. G., Avishai, E. S. and Baram, T. Z., Corticotropin releasing factor receptor type II (CRF2) messenger ribonucleic acid levels in the hypothalamic ventromedial nucleus of the infant rat are reduced by maternal deprivation, Endocrinology, 138 (1997) 5048-5051.

Ekman, R., Servenius, B., Castro, M. G., Lowry, P. J., Cederlund, A. S., Bergman, O. and Sjogren, H. O., Biosynthesis of corticotropin-releasing hormone in human T-lymphocytes, J. Neuroimmunol., 44 (1993) 7-13.

File, S. E., The contribution of behavioural studies to the neuropharmacology of anxiety, Neuropharmacology 26 (1987) 877-886.

Florin, S., Suaudeau, C., Meunier, J. C. and Costentin, J., Nociceptin stimulates locomotion and exploratory behaviour in mice, Euro. J. Pharmacol., 317 (1996)9-13.

Fuhlendorff, J., U. Gether, L. Aakerlund, N. Langeland-Johansen, H. Togerson, S. G F. Melberg, U. B.

Olsen, O. Thastrup, and T. W. Schwartz 31 34 (1990), [LeU, Pro I Neuropeptide Y: A specific Yj receptor agonist, Proc. Natl. Acad. Sci. 87, 182-186.

Gaveriaux, R. C., Matthes, H. W., Peluso, J. and Kieffer, B. L., Abolition of morphine-immunosuppression in mice lacking the mu-opioid receptor gene, Proc. Natl. Acad. Sci. U.S.A., 95 (1998) 6326-6330.

Gehlert, D. R., Multiple receptors for the pancreatic polypeptide (PP-fold) family: physiological implications, Proc. Soc. Exp. Biol. Med., 218 (1998) 7-22.

Gerald, C., Walker, M, W., Criscione, L., Gustafson, E. L., Batzi-Hartmann, C., Smith, K. E,, Vaysse, P., Durkin, M. M., Laz. T. M., Linerneyer, D. L., Schaffhauser, A. O. Whitebread, S., Hofbauer, K. G., Taber, R. I., Branchek, T, A. and Weinshank, R. L. (1996). A receptor subtype involved in neuropeptide-Y-induced food intake. Nature, 382, 168-171.

Gerald, C., Walker, M. W., Vaysse, P. J. -J, He, C., Branchek, T. A. and Weinshank. R. L. (1995) J. Biol. Chem., 270, 26758-26761. Expression cloning and pharmacological characterisation of a human hippocampal neuropeptide Y/peptide YY Y2 receptor subtype.

Gosnell, B. A., Levine, A. S. and Morley, J. E., The stimulation of food intake by selective agonists of mu, kappa and delta opioid receptors, Life Sci., 38 (1986) 1081-1088.

Grosskreutz, C. L. and Brody, M. J., Regional hemodynamic responses to central administration of corticotropin-releasing factor (CRF), Brain Res., 442 (1988) 363-367.

Grouzmann, E., Buclin, T., Martire, M., Cannizzaro, C., Dorner, B., Razaname, A. and Mutter, M., Characterization of a selective antagonist of neuropeptide Y at the Y2 receptor. Synthesis and pharmacological evaluation of a Y2 antagonist, J. Biol. Chem., 272 (1997) 7699-7706.

Grundemar, L., Grundstrom, N., joahansson, I. G. M., Andersson, R. G. G. and Hakanson, R. (1990) Suppression by neuropeptide Y of capsaicin-sensitive sensory nerve-mediated contraction in guinea-pig airways. Br. J. Pharmacol., 99, 473476.

Grundemar, L., Wahlestedt, C. and Wang, Z. Y. (1993) Neuropeptide Y suppresses the neurogenic inflammatory response in the rabbit eye; mode of action. Regul. Pept., 43, 57-64.

Grunditz, T., Uddman, R. and Sundler, F. (1994) Origin and peptide content of nerve fibers in the nasal mucosa of rats. Anat. Embryol. 189, 327-337.

Gue, M., Junien, J. L., Reeve-J R, J., Rivier, J., Grandt, D. and Tache, Y., Reversal by NPY, PYY and 3-36 molecular forms of NPY and PYY of intracisternal CRF-induced inhibition of gastric acid secretion in rats, Br. J. Pharmacol., 118 (1996) 237-242.

Heilig, M. and Murison, R., Intracerebroventricular neuropeptide Y suppresses open field and home cage activity in the rat, Regul. Pept., 19 (1987) 221-231.

Heilig, M., B. Söderpalm, J. A. Engel and E. Widerlöv, (1989), Centrally administered neuropeptide Y (NPY) produces anxiolytic-like effects in animal anxiety models, Psychopharmacology, 98, 524-529.

Heilig, M., McLeod, S., Brot, M., Heinrichs, S. C., Menzaghi, F., Koob, G. F. and Britton, K. T., Anxiolytic-like action of neuropeptide Y: mediation by Y1 receptors in amygdala, and dissociation from food intake effects, Neuropsychopharmacology., 8 (1993) 357-363.

Heilig, M., Soderpalm, B., Engel, J. A. and Widerlov, E., Centrally administered neuropeptide Y (NPY) produces anxiolytic-like effects in animal anxiety models, Psychopharmacology Berl., 98 (1989) 524-529.

Heinrichs, S. C., Lapsansky, J., Behan, D. P., Chan, R. K. , Sawchenko, P. E., Lorang, M., Ling, N., Vale, W. W. and De-Souza, E. B., Corticotropin-releasing factor-binding protein ligand inhibitor blunts excessive weight gain in genetically obese Zucker rats and rats during nicotine withdrawal, Proc. Natl. Acad. Sci. U. S. A., 93 (1996) 15475-15480.

Heinrichs, S. C., Pich, E. M., Miczek, K. A., Britton, K. T. and Koob, G. F., Corticotropin-releasing factor antagonist reduces emotionality in socially defeated rats via direct neurotropic action, Brain Res., 581 (1992) 190-197.

Herz, A., Opioid reward mechanisms: a key role in drug abuse?, Can. J. Physiol. Pharmacol., 76 (1998) 252-258.

Herzog, H., Y. J. Hort, H. J. Ball, G. Hayes, J. Shine, and L. A. Selbie (1992), Cloned human neuropeptide Y receptor couples to two different second messenger systems, Proc. Natl. Acad. Sci. U.S.A. 89, 5794-5798.

Hoehe, M. and Duka, T., Opiates increase plasma catecholamines in humans, Psychoneuroendocrinology., logy., 18 (1993) 141-148.

Hoffman, K. E., Maslonek, K. A., Dykstra, L. A. and Lysle, D. T., Effects of central administration of morphine on immune status in Lewis and Wistar rats. In B. M. Sharp, T. K. Eisenstein, J. J. Madden and H. Friedman (Eds.) The brain immune axis and substance abuse, Plenum Press, New York, 1995,pp. 155-159.

Horvath, T. L., Naftolin, F., Kalra, S. P. and Leranth, C., Neuropeptide-Y innervation of beta-endorphin-containing cells in the rat mediobasal hypothalamus: a light and electron microscopic double immunostaining analysis [published erratum appears in Endocrinology February 1996; 137(2):532], Endocrinology, 131 (1992) 2461-2467.

Jankovic, B. D. and Radulovic, J., Enkephalins, brain and immunity: modulation of immune responses by methionine-enkephalin injected into the cerebral cavity, Int. J. Neurosci., 67 (1992) 241-270.

Jenck, F., Moreau, J. L., Martin, J. R., Kilpatrick, G. J., Reinscheid, R. K., Monsma-F J, J., Nothacker, H. P. and Civelli, O., Orphanin FQ acts as an anxiolytic to attenuate behavioral responses to stress, Proc. Natl. Acad. Sci. U.S.A., 94 (1997) 14854-14858.

Kalra, P. S., Norlin, M. and Kalra, S. P., Neuropeptide Y stimulates beta-endorphin release in the basal hypothalamus: role of gonadal steroids, Brain Res., 705 (1995) 353-356.

Karalis, K., Muglia, L. J., Bae, D., Hilderbrand, H. and Majzoub, J. A., CRH and the immune system, J. Neuroimmunol., 72 (1997) 131-136.

Kask, A., L. Rägo and J. Harro, (1998), NPY Y1 receptors in the dorsal periaqueductal gray matter regulate anxiety in the social interaction test, Neuroreport, 9, 2713-2716.

Kask, A., Rago, L. and Harro, J., Anxiolytic-like effect of neuropeptide Y (NPY) and NPY13-36 micro-injected into vicinity of locus coeruleus in rats, Brain Res., 788 (1998) 345-348.

Kiritsy, R. J., Appel, N. M., Bobbitt, F. G. and Van-Loon, G. R., Effects of mu-opioid receptor stimulation in the hypothalamic paraventricular nucleus on basal and stress-induced catecholamine secretion and cardiovascular responses, J. Pharmacol. Exp. Ther., 239 (1986) 814-822.

Kiritsy, R. J., Marson, L. and Van-Loon, G. R., Sympathoadrenal, cardiovascular and blood gas responses to highly selective mu and delta opioid peptides, J. Pharmacol. Exp. Ther., 251 (1989) 1096-1103.

Konig, M., Zimmer, A. M., Steiner, H., Holmes, P. V., Crawley, J. N., Brownstein, M. J. and Zimmer, A., Pain responses, anxiety and aggression in mice deficient in pre-proenkephalin, Nature, 383 (1996) 535-538.

Koob, G. F. and Bloom, F. E., Corticotropin-releasing factor and behavior, Fed. Proc., 44 (1985) 259-263.

Kotz, C. M., Grace, M. K., Billington, C. J. and Levine, A. S., The effect of norbinaltorphimine, beta-funaltrexamine and naltrindole on NPY-induced feeding, Brain Res., 631 (1993) 325-328.

Krepela E, Kraml J, Vicar J, Kadlecova L, Kasafirek E: Dipeptidyl peptidase IV hydrolyses Gastric Inhibitory Polypeptide, Glucagon-like Peptide-1 (7-36)amide, Peptide Histidine Methionin and is Responsible for their Degradation in Human Serum, Eur. J. Biochem. 214, 829-835 (1993)

Lacroix, J. S., Ulman, L. G. and Potter, E. K. (1994) Modulation by neuropeptide Y of parasympathetic nerve-evoked nasal vasodilitation via Y2 prejunctional receptor. Br. J. Pharmacol., 113, 479-484.

Lambert, P. D., Wilding, J. P., al-Dokhayel, A. A., Gilbey, S. G. and Bloom, S. R., The effect of central blockade of kappa-opioid receptors on neuropeptide Y-induced feeding in the rat, Brain Res., 629 (1993) 146-148.

Leu, S. J. and Singh, V. K., Modulation of natural killer cell-mediated lysis by corticotropin-releasing neurohormone, J. Neuroimmunol., 33 (1991) 253-260.

Levine, A. S. and Billington, C. J., Opioids. Are they regulators of feeding?, Ann. N.Y. Acad. Sci., 575 (1989) 209-219.

Locke, K. W. and Holtzman, S. G., Behavioral effects of opioid peptides selective for mu or delta receptors. I. Morphine-like discriminative stimulus effects, J. Pharmacol. Exp. Ther., 238 (1986) 990-996.

Loh, H. H., Liu, H. C., Cavalli, A., Yang, W., Chen, Y. F. and Wei, L. N., mu Opioid receptor knockout in mice: effects on ligand-induced analgesia and morphine lethality, Brain Res. Mol. Brain Res., 54 (1998) 321-326.

Lu, X. -Y., Gunn, T. M., Shieh, K. -R., Barsh, G. S., Akil, H., Watson, S. J., Distribution of Mahogany/Attractin mRNA in the rat central nervous system. FEBS Letters 462 (1999), 101-107.

Lundberg, J. M., Pharmacology of cotransmission in the autonomic nervous system: Intergrative aspects on amines, neruopeptides, adenosine triphosphaste, amino acids and nitric oxide, Pharmacol. Rev., 48 (1996) 113-178.

Lundberg, J. M., Hensen, A., Larsson, 0., Rudehill, A., Saria, A & Fredholm B. B., (1988). Neuropeptide Y receptor in pig spleen; binding characteristics, reduction of cAMP formation and calcium antagonist inhibition of vasoconstriction. Eur. J. Pharmacol. Vol. 45; 21-29

Lysle, D. T., Hoffman, K. E. and Dykstra, L. A., Evidence for the involvement of the caudal region of the periaqueductal gray in a subset of morphine-induced alterations of immune status, J. Pharmacol. Exp. Ther., 277 (1996) 1533-1540.

Makino, S., Takemura, T., Asaba, K., Nishiyama, M., Takao, T. and Hashimoto, K., Differential regulation of type-1 and type-2alpha corticotropin-releasing hormone receptor mRNA in the hypothalamic paraventricular nucleus of the rat, Brain Res. Mol. Brain Res., 47 (1997) 170-176.

Mamiya, T., Noda, Y., Nishi, M., Takeshima, H. and Nabeshima, T., Enhancement of spatial attention in nociceptin/orphanin FQ receptor-knockout mice, Brain Res., 783 (1998) 236-240.

Manabe, T., Noda, Y., Mamiya, T., Katagiri, H., Houtani, T., Nishi, M., Noda, T., Takahashi, T., Sugimoto, T., Nabeshima, T. and Takeshima, H., Facilitation of long-term potentiation and memory in mice lacking nociceptin receptors, Nature, 394 (1998) 577-581.

Matthes, H. W., Maldonado, R., Simonin, F., Valverde, 0., Slowe, S., Kitchen, I., Befort, K., Dierich, A., Le-Meur, M., Dolle, P., Tzavara, E., Hanoune, J., Roques, B. P. and Kieffer, B. L., Loss of morphine-induced analgesia, reward effect and withdrawal symptoms in mice lacking the mu-opioid-receptor gene [see comments], Nature, 383 (1996) 819-823.

May, C. N., Dashwood, M. R., Whitehead, C. J. and Mathias, C. J., Differential cardiovascular and respiratory responses to central administration of selective opioid agonists in conscious rabbits: correlation with receptor distribution, Br. J. Pharmacol., 98 (1989) 903-913.

Mellado, M. L., Gibert, R. J., Chover, A. J. and Mico, J. A., Effect on nociception of intracerebroventricular administration of low doses of neuropeptide Y in mice, Life Sci., 58 (1996) 2409-2414.

Mellon, R. D. and Bayer, B. M., Evidence for central opioid receptors in the immunomodulatory effects of morphine: review of potential mechanism(s) of action, J. Neuroimmunol., 83 (1998) 19-28.

Mellon, R. D. and Bayer, B. M., Role of central opioid receptor subtypes in morphine-induced alterations in peripheral lymphocyte activity, Brain Res., 789 (1998) 56-67.

Menzaghi, F., Heinrichs, S. C., Pich, E. M., Tilders, F. J. and Koob, G. F., Functional impairment of hypothalamic corticotropin-releasing factor neurons with immunotargeted toxins enhances food intake induced by neuropeptide Y, Brain Res., 618 (1993) 76-82.

Menzaghi, F., Howard, R. L., Heinrichs, S. C., Vale, W., Rivier, J. and Koob, G. F., Characterization of a novel and potent corticotropin-releasing factor antagonist in rats, J. Pharmacol. Exp. Ther., 269 (1994) 564-572.

Mercer, M. E. and Holder, M. D., Food cravings; endogenous opioid peptides, and food intake: a review, Appetite., 29 (1997) 325-352.

Meunier. J. C., Mollreau, C., Toll, L., Suaudeau, C., Moisand, C., Alvinerie, P., Butour, J. L ., Guillemot, J. C., Ferrara, P., Monsarrat, B. and et, a., Isolation and structure of the endogenous agonist of opioid receptor-like ORL1 receptor [see comments], Nature, 377 (1995) 532-535.

Millan, M. J., Kappa-opioid receptors and analgesia, Trends. Pharmacol. Sci., 11 (1990) 70-76.

Minami, M. and Satoh, M., Molecular biology of the opioid receptors: structures, functions and distributions, Neurosci. Res., 23 (1995) 121-145.

Mogil, J. S., Grisel, J. E., Reinscheid, R. K., Civelli, O., Belknap, J. K. and Grandy, D. K., Orphanin FQ is a functional anti-opioid peptide, Neuroscience, 75 (1996) 333-337.

Mollereau, C., Parmentier, M., Mailleux, P., Butour, J. L., Moisand, C., Chalon, P., Caput, D., Vassart, G. and Meunier, J. C., ORL1, a novel member of the opioid receptor family. Cloning, functional expression and localization, FEBS Lett., 341 (1994) 33-38.

Motta, V. and Brandao, M. L., Aversive and antiaversive effects of morphine in the dorsal periaqueductal gray of rats submitted to the elevated plus-maze test, Pharmacol. Biochem. Behav., 44 (1993) 119-125.

Nässel, Dr., Mentlein, R., Bollner, T., Karlsson, A., Proline-specific dipeptidyl peptidase activity in the cockroach brain and intestine: partial characterization, distribution, and inactivation of tachykinin-related peptides, J Comp Neurol, 418 (2000) 81-92

Nishi, M., Houtani, T., Noda, Y., Mamiya, T., Sato, K., Doi, T., Kuno, J., Takeshima, H., Nukada, T., Nabeshima, T., Yamashita, T., Noda, T. and Sugimoto, T., Unrestrained nociceptive response and disregulation of hearing ability in mice lacking the nociceptin/orphaninFQ receptor, EMBO J., 16 (1997) 1858-1864.

Noble, F., Smadja, C., Valverde, O., Maldonado, R., Coric, P., Turcaud, S., Foumie, Z. M. and Roques, B. P., Pain-suppressive effects on various nociceptive stimuli (thermal, chemical, electrical and inflammatory) of the first orally active enkephalin-metabolizing enzyme inhibitor RB 120, Pain, 73 (1997) 383-391.

Noda, Y., Mamiya, T., Nabeshima, T., Nishi, M., Higashioka, M. and Takeshima, 11., Loss of antinociceoption induced by naloxone benzoylhydrazone in nociceptin receptor-knockout mice, J. Biol. Chem., 273 (1998) 18047-18051.

Novak, J. E., Gomez-Flores, R., Calderon, S. N., Rice, K. C. and Weber, R. J., Rat natural killer cell, T cell and macrophage functions after intracerebroventricular injection of SNC80, J. Pharmacol. Exp. Ther., 286 (1998) 931-937.

Olson, G. A., Olson, R. D. and Kastin, A. J., Endogenous opiates: 1995, Peptides, 17 (1996) 1421-1466.

Ono, N., Lumpkin, M. D., Samson, W. K., McDonald, J. K. and McCann, S. M., Intrahypothalamic action of corticotrophin-releasing factor (CRF) to inhibit growth hormone and LH release in the rat, Life Sci., 35 (1984) 1117-1123.

Pechnick, R. N., Effects of opioids on the hypothalamo-pituitary-adrenal axis, Annu. Rev. Pharmacol. Toxicol., 33 (1993) 353-382.

Pedrazzini, T., Seydoux, J., Kunstner, P., Aubert, J. F., Grouzmann, E., Beermann, F. and Brunner, H. R., Cardiovascular response, feeding behavior and locomotor activity in mice lacking the NPY Y1 receptor [see comments], Nat. Med., 4 (1998) 722-726.

Pfeiffer, A., Brantl, V., Herz, A. and Emrich, H. M., Psychotomimesis mediated by kappa opiate receptors, Science, 233 (1986) 774-776.

Pomonis, J. D., Billington, C. J. and Levine, A. S., Orphanin FQ, agonist of orphan opioid receptor ORL1, stimulates feeding in rats, Neuroreport., 8 (1996) 369-371.

Potter, E. K. and M. J, D. McCloskey, (1992), [Leu 3′, Leu 34 1 neuropeptide Y, a 45 selective functional agonist at neuropeptide Y receptors in anaesthetised rats, Neurosci. Lett. 134, 183-186,

Potter, E X, J. Fuhlendorff and T. W. Schwartz (1991), [Pro34 J neuropeptide Y selectively identifies postjunctional-mediated actions of neuropeptide Y in vivo in rats and dogs, Eur. J. Pharmacol. 193, 15-19.

Potter, E X, Mitchell, L., McCloskey, M. J., Tseng, A., Goodman, A. E., Shine, J. and McCloskey, D. I. (1989) Pre-and postjunctional actions of neuropeptide Y and related peptides. Regul. Pept. 25, 167-177.

Privette, T. H. and Terrian, D. M., Kappa opioid agonists produce anxiolytic-like behavior on the elevated plus-maze, Psychopharmacology Berl., 118 (1995) 444-450.

Radulovic, J. and Jankovic, B. D., Opposing activities of brain opioid receptors in the regulation of humoral and cell-mediated immune responses in the rat, Brain Res., 661 (1994) 189-195.

Radulovic, J., Miljevic, C., Djergovic, D., Vujic, V., Antic, J., von-Horsten, S. and Jankovic, B. D., Opioid receptor-mediated suppression of humoral immune response in vivo and in vitro: involvement of kappa opioid receptors, J. Neuroimmunol., 57 (1995) 55-62.

Rassnick, S., Heinrichs, S. C., Britton, K. T. and Koob, G. F., Microinjection of a corticotropin-releasing factor antagonist into the central nucleus of the amygdala reverses anxiogenic-like effects of ethanol withdrawal, Brain Res., 605 (1993) 25-32.

Reinscheid, R. K., Nothacker, H. P., Bourson, A., Ardati, A., Henningsen, R. A., Bunzow, J. R., Grandy, D. K., Langen, H., Monsma-F J, J. and Civelli, O., Orphanin FQ: a neuropeptide that activates an opioidlike G protein-coupled receptor, Science, 270 (1995) 792-794.

Rioux, F., H. Bachelard, J. C. Martel and S. St.-Piere, (1986), The vasoconstrictor effect of neuropeptide Y and related peptides in the guinea pig isolated heart, Peptides, 7, 27-31.

Risdahl, J. M., Khanna, K. V., Peterson, P. K. and Molitor, T. W., Opiates and infection, J. Neuroimmunol., 83 (1998) 4-18.

Rivier, C., Rivier, J., Mormede, P. and Vale, W., Studies of the nature of the interaction between vasopressin and corticotropin-releasing factor on adrenocorticotropin release in the rat, Endocrinology, 115 (1984) 882-886.

Rivier, C. L. and Plotsky, P. M., Mediation by corticotropin releasing factor (CRF) of adenohypophysial hormone secretion, Annu. Rev. Physiol., 48 (1986) 475-494.

Rossi, G. C., Leventhal, L. and Pasternak, G. W., Naloxone sensitive orphanin. FQ-induced analgesia in mice, Eur. J. Pharmacol., 311 (1996) R7-R8

Sandin, J., Georgieva, J., Schott, P. A., Ogren, S. O. and Terenius, L., Nociceptin/orphanin FQ microinjected into hippocampus impairs spatial learning in rats, Euro. J. Neurosci., 9 (1997) 194-197.

Saperstein, A., Brand, H., Audhiya, T., Nabriski, D., Hutchinson, B., Rosenzweig, S. and Hollander, C. S., Interleukin 1 beta mediates stress-induced immunosuppression via corticotropin-releasing factor, Endocrinology, 130 (1992) 152-158.

Satoh, M. and Minami, M., Molecular pharmacology of the opioid receptors, Pharmacol. Ther., 68 (1995) 343-364.

Schwartz, T. W., J. Fuhlendorff, H, Langeland, J. C. T6gerson, S. P. Sheikh, (1989), in Neuropeptide Y—XIV Nobel Symposium, ed: V. Mutt; T. H6kfelt, K. Fuxe and J. M. Lundberg, Raven, N.Y. pp 143.

Shavit, J. (1991) Stress-induced immune modulation in animals: opiates and endogenous opioid peptides. In: R. Ader, D. L. Felten and N. Cohen (Eds. ), Psychoneuroimmunology, Vol. Academic Press, San Diego, pp. 789-804.

Shavit, Y., Depaulis, A., Martin, F. C., Terman, G. W., Pechnick, R. N., Zane, C. J., Gale, R. P. and Liebeskind, J. C., Involvement of brain opiate receptors in the immune-suppressive effect of morphine, Proc. Natl. Acad. Sci. U.S.A., 83 (1986) 7114-7117.

Sheikh, S. P., R. HAkanson and T. W. Schwartz, (1989), Yj and Y2receptors for neuropeptide Y, FEBS Lett. 245, 209-214.

Shippenberg, T. S., Bals, K. R. and Herz, A., Motivational properties of opioids: evidence that an activation of delta-receptors mediates reinforcement processes, Brain Res., 436 (1987) 234-239.

Simonin, F., Valverde, O., Smadja, C., Slowe, S., Kitchen, I., Dierich, A., Le-Meur, M., Roques, B. P., Maldonado, R. and Kieffer, B. L., Disruption of the kappa-opioid receptor gene in mice enhances sensitivity to chemical visceral pain, impairs pharmacological actions of the selective kappa-agonist U-50,488H and attenuates morphine withdrawal, EMBO J., 17 (1998) 886-897.

Sora, I., Funada, M. and Uh1, G. R., The mu-opioid receptor is necessary for [D-Pen2,D-Pen5]enkephalin-induced analgesia, Eur. J. Pharmacol., 324 (1997) R1-R2

Stanley, B. G. and Leibowitz, S. F., Neuropeptide Y injected in the paraventricular hypothalamus: a powerful stimulant of feeding behavior, Proc. Natl. Acad. Sci. U.S.A., 82 (1985) 3940-3943.

Stanley, B. G., Lanthier, D., Chin, A. S. and Leibowitz, S. F., Suppression of neuropeptide Y-elicited eating by adrenalectomy or hypophysectomy: reversal with corticosterone, Brain Res., 501 (1989) 32-36.

Stefano, G. B., Salzet, B. and Fricchione, G. L., Enkelytin and opioid peptide association in invertebrates and vertebrates: immune activation and pain, Immunol. Today, 19 (1998) 265-268.

Tatemoto, K., Cariquist, M. and Mutt, V., Neuropeptide Y—a novel brain peptide with structural similarities to peptide YY and pancreatic polypeptide, Nature, 296 (1982) 659-660.

Tatemoto, K., Neuropeptide Y: complete amino acid sequence of the brain peptide, Proc. Natl. Acad. Sci. U.S.A., 79 (1982) 5485-5489.

Tejedor, R. P., Costela, C. and Gibert, R. J., Neonatal handling reduces emotional reactivity and susceptibility to learned helplessness. Involvement of catecholaminergic systems, Life Sci., 62 (1998) 37-50.

Tian, M., Broxmeyer, H. E., Fan, Y., Lai, Z., Zhang, S., Aronica, S., Cooper, S., Bigsby, R. M., Steinmetz, R., Engle, S. J., Mestek, A., Pollock, J. D., Lehman, M. N., Jansen, H. T., Ying, M., Stambrook, P. J., Tischfield, J. A. and Yu, L., Altered hematopoiesis, behavior, and sexual function in mu opioid receptor-deficient mice, J. Exp. Med., 185 (1997) 1517-1522.

Tortella, F. C. and DeCoster, M. A., Kappa opioids: therapeutic considerations in epilepsy and CNS injury, Clin. Neuropharmacol., 17 (1994) 403416.

Tsuda, M., Suzuki, T., Misawa, M. and Nagase, H., Involvement of the opioid system in the anxiolytic effect of diazepam in mice, Eur. J. Pharmacol.,307 (1996) 7-14.

Uehara, Y., Shimizu, H., Ohtani, K., Sato, N. and Mori, M., Hypothalamic corticotropin-releasing hormone is a mediator of the anorexigenic effect of leptin, Diabetes, 47 (1998) 890-893.

Vaughan, J., Donaldson, C., Bittencourt, J., Perrin, M. H., Lewis, K., Sutton, S., Chan, R., Tumbull, A. V., Lovejoy, D., Rivier, C. and et, a., Urocortin, a mammalian neuropeptide related to fish urotensin 1 and to corticotropin-releasing factor [see comments], Nature, 378 (1995) 287-292.

Wahlestedt, C. and Reis, D. J., Neuropeptide Y-related peptides and their receptors--are the receptors potential therapeutic drug targets?, Annu. Rev. Pharmacol. Toxicol., 33 (1993) 309-352.

Wahlestedt, C., N. Yanaihara and R. H. Akanson, (1986), Evidence for different pre- and post-junctional receptors for neuropeptide Y and related peptides, Regul. Pep. 13, 307-318.

Wahlestedt, C., Pich, E. M., Koob, G. F., Yee, F. and Heilig, M., Modulation of anxiety and neuropeptide Y-Y1 receptors by antisense oligodeoxynucleotides, Science, 259 (1993) 528-531.

Wettstein, J. G., Earley, B. and Junien, J. L., Central nervous system pharmacology of neuropeptide Y, Pharmacol. Ther., 65 (1995) 397-414.

Xu, X. J., Hao, J. X. and Wiesenfeld, H. Z., Nociceptin or antinociceptin: potent spinal antinociceptive effect of orphanin FQ/nociceptin in the rat, Neuroreport., 7 (1996) 2092-2094.

Zadina, J. E., Hackler, L., Ge, L. J. and Kastin, A. J., A potent and selective endogenous agonist for the mu-opiate receptor [see comments], Nature, 386 (1997) 499-502.

Zhao, X. J., Hoheisel, G., Schauer, J. and Bornstein, S. R., Corticotropin-releasing hormone-binding protein and its possible role in neuroendocrinological research, Horm. Metab. Res., 29 (1997) 373-378.

Zhu, Y, and Im, W. B., Block of sodium channel current by anticonvulsant U-54494A in mouse neuroblastoma cells, J. Pharmacol. Exp. Ther., 260 (1992) 110-116.

1. Use of an inhibitor of attractin or of an attractin isoform for the production of a medicament for modulating behavioral and/or neurological responsiveness to stress including anxiety.

2. Use according to claim 1 for the production of a medicament for the reduction of degradation of the endogenous, CNS-localized neuropeptide Y (NPY) and other substrates sharing similar properties.

3. Use according to any one of the preceding claims for the production of a medicament for the treatment of psychosomatic, depressive and neuropsychiatric diseases.

4. Use according to claim 3, characterized in that the psychosomatic, depressive and neuropsychiatric diseases are selected from the group consisting of anxiety disorders, depression, insomnia, chronic fatigue, schizophrenia, epilepsy, eating disorders, spasm and chronic pain.

5. Use according to any one of the preceding claims, characterized in that the inhibitors are used in combination with neuropeptide Y.

6. Use according to any one of the preceding claims, characterized in that the inhibitors are present in a physiologically compatible drug delivery vehicle.

7. Use according to any one of the preceding claims, characterized in that the inhibitors are formulated as prodrugs of the inhibitors.

8. Use according to any one of the preceding claims, characterized in that the inhibitors are used parenterally, enterally, orally, by inhalation or as a suppository.
